(19) United States
(12) Patent Application Publication
(10) Pub. No.: US 2007/0082944 A1
(43) Pub. Date: Apr. 12, 2007

(54) Antimicrobial compositions and methods of use

(75) Inventors: Paul Stapleton, London (GB); Shinich Uesato, Kyoto (JP); Peter W. Taylor, West Sussex (GB); Yukihiko Hara, Tokyo (JP)

Correspondence Address: Hani Z. Sayed, Esq.;c/o Rutan & Tucker, LLP, 611 ANTON BLVD, SUITE 1400, COSTA MESA, CA, 92626, US

(73) Assignee: Robert Fish, Costa Mesa, CA (US)

(21) Appl. No.: 10/569,526

(22) PCT Filed: Sep. 12, 2003
(86) PCT No.: PCT/US03/28750

§ 371 Date: Nov. 13, 2006

(51) Int. Cl. A61K 31/353; G06Q 50/00

(52) U.S. Cl. 514/456; 705/2

(57) ABSTRACT

A catechin is modified in at least one position (most preferably in the 3-position of the C-ring) to increase its lipophilicity. Contemplated catechins are demonstrated to have significantly improved antibacterial properties, likely due to catastrophic membrane damage.

### FIELD OF THE INVENTION

The field of the invention is antimicrobial agents and compositions, and especially those including modified catechins.

### BACKGROUND OF THE INVENTION

While use of antibiotics allowed physicians to successfully treat numerous diseases over the last decades, almost all bacteria treated with antibiotics have developed at least some degree of resistance against these drugs. For example, various strains of multi-drug resistant Staphylococcus aureus are commonly found in hospitals.

S. aureus is a gram-positive, pyogenic, and opportunistic pathogen, known to be the etiologic agent for a range of infections, including sepsis, pneumonia, endocarditis and soft tissue infections. The bacterial cell carries protein A on the surface of the cell wall to bind potentially neutralizing antibodies, and coagulase produced by the bacterium often correlates with virulence. Of particular concern is a group of S. aureus strains that is resistant to substantially all antibiotics of the beta-lactam class (a.k.a. MRSA: Methicillin Resistant S. aureus), and especially including cephalosporins. Beta-lactam antibiotics bind to bacterial proteins called “Penicillin Binding Proteins” (PBPs). In MRSA, PBP2 and PBP2′ are typically key to resistance in MRSA (however, PBP2′ is altered to such an extent that beta-lactam antibiotics bind only poorly to it). In addition, most S. aureus strains secrete beta-lactamase, which hydrolyzes various beta-lactam antibiotics (e.g., benzylpenicillin, or ampicillin; other beta-lactam antibiotics, including such as methicillin or cephalothin are not hydrolyzed by the beta-lactamase under most circumstances).

MRSA infections can be treated with glycopeptides (e.g., vancomycin). While such antibiotics overcome at least some of the problems with resistance, glycopeptides are often expensive and potentially toxic. Worse yet, resistance to the glycopeptides has emerged in closely related bacteria, and significant resistance has recently been reported in MRSA in one patient in the US (several cases of intermediate resistance were already reported earlier).

Remarkably, specific preparations of tea, and especially green tea have recently been shown to exhibit remarkable antibacterial effect against MRSA. For example, Shimamura et al. describe in U.S. Pat. No. 5,358,713 use of tea and tea polyphenols as agents to prevent or reduce transmission of MRSA from one patient to another patient. Similarly, Hamilton-Miller describes in U.S. Pat. No. 5,879,683 use of tea extracts to restore sensitivity of MRSA to beta-lactam antibiotics. In yet another example, Shimamura describes in EP 0443090 that an extract of tea at a concentration of about 0.2-2.0 g/100 ml is capable of preventing the growth of a number of types of bacteria, including some strains of MRSA. While such preparations indeed have unexpected antibacterial effects, various problems nevertheless remain. Among other things, relatively high concentrations and dosages are often required to reach at least somewhat satisfactory effect. Moreover, m many cases, the catechin only restores sensitivity against a beta-lactam antibiotic and therefore, coadministration with an antibiotic is required.

Further biological activities for tea extracts, and especially tea catechins are published in various sources. For example, 3-O-acyl-(−)-epigallocatechin were reported to have anti-tumor promoting activities at the Twentieth International Conference on Polyphenols (in Freising-Weihenstephan; Germany; Sep. 11-15, 2000 by S. Uesato, K. Yutaka, H. Yukihiko,T. Harukuni, M. Okuda, T. Mukainaka, H. Nishino). However, the mechanism of such action is poorly understood, and further investigation is needed to optimize treatment results.

Therefore, while various compositions and methods for catechins are known in the art, all or almost all of them suffer from one or more disadvantages. Thus, there is still a need to provide improved compositions and methods for catechins, especially for antimicrobial use.

### SUMMARY OF THE INVENTION

The present invention is directed to compositions and methods of modified catechins in which the lipophilicity of a catechin increased by adding a lipophilic substituent to one or more positions in the catechin. Such modified catechins exhibit superior antibacterial properties, including antibacterial activity against MRSA.

Therefore, in one aspect of the inventive subject matter, a pharmaceutical composition includes a modified catechin according to Formula 1

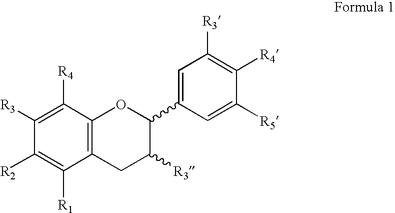

wherein R₁, R₂, R₃, R₄, R₃′, R₄′, and R₅′ are independently H, OH, or M, wherein R₃″ is H, OH, an optionally substituted phenyl, or M, with the proviso that at least one of R₁, R₂, R₃, R₄, R₃′, R₄′, R₅′, and R₃″ is M; wherein M is OC(O)R, OC(S)R, OC(NH)R, OR, or R, wherein R is optionally substituted alkyl, alkenyl, alkynyl, alkaryl, or aryl; and wherein the modified catechin is present at a concentration effective to reduce bacterial growth in a body compartment when administered to the body compartment.

Particularly preferred modified catechins will include those in which the 3-hydroxy group of the C-ring (i.e., the tetrahydropyran ring of the catechin scaffold) is modified with a lipophilic group, preferably with an OC(O)R group, and most preferably with OC(O)CH₂(CH₂)₅CH₃ or OC(O)CH₂(CH₂)₇CH₃. The R₁, R₃, R₃′, and R₄′ groups in such molecules are preferably OH, while the R₂ and R₄ groups are preferably H. In further preferred aspects, the modified catechin is an isomerically and optically pure compound (most preferably (+)).

In further preferred aspects of such pharmaceutical compositions, the bacterial growth is that of a gram-positive bacterium (e.g., S. aureus, optionally resistant to a beta-lactam antibiotic and/or cephalosporins), and the body compartment comprises the skin of a patient and wherein the administration is topical administration. Administration of such modified catechins is contemplated to damage the bacterial membrane (preferably the cellular lipid bilayer membrane), and it is further contemplated that the modified catechin increases sensitivity of a methicillin resistant S. aureus toward a beta-lactam antibiotic no more than 2-fold.

Consequently, in another aspect of the inventive subject matter, a method of reducing growth of a bacterium may include a step in which the bacterium is contacted with a modified catechin having a structure according to Formula 1 (supra), and with respect to further preferred aspects of the modified catechin and its applications, the same considerations as above apply.

Therefore, where contemplated catechins are commercially exploited, the inventors also contemplate a method of marketing in which a product is provided that includes the modified catechin according to Formula 1 (supra). In another step, it is advertised that the product reduces bacterial growth. Especially preferred products include cosmetic formulations, cleaning formulations, and/or pharmaceutical formulations, while preferred manners of advertising include providing printed information suggesting or describing reduction of bacterial growth, and/or providing televised information suggesting or describing reduction of bacterial growth.

Various objects, features, aspects and advantages of the present invention will become more apparent from the following detailed description of preferred embodiments of the invention, along with the accompanying drawing.

### BRIEF DESCRIPTION OF THE DRAWINGS

FIG. 1 is a graph depicting the antimicrobial effect of a predetermined dose of selected modified catechins on a methicillin resistant strain of S. aureus in the presence of rising doses of Oxacillin.

FIG. 2 is a graph depicting the dose-dependent antimicrobial effect of selected modified catechins on a methicillin resistant strain of S. aureus.

FIG. 3 is a graph depicting the dose-dependent antimicrobial effect of an exemplary modified catechin on various strains of S. aureus.

FIG. 4 is a graph depicting the dose-dependent antimicrobial effect of epicatechin gallate on S. aureus strain EMRSA-16.

FIG. 5 is a graph depicting the dose-dependent antimicrobial effect of octanoyl catechin on S. aureus strain EMRSA-16.

FIG. 6A is an electron micrograph depicting S. aureus treated with epicatechin gallate.

FIG. 6B is a electron micrograph depicting S. aureus treated with 3-O-octanoyl-(−)-epicatechin.

### DETAILED DESCRIPTION

The inventors surprisingly discovered that various lipophilic modifications to numerous isoflavonoids can be made to give modified catechins, wherein such modified catechins exhibit a significantly improved antibacterial activity. In one particularly preferred example, the inventors discovered that the antibacterial activity of epicatechin gallate can be dramatically increased when the 3-substituent on the C-ring (here: OC(O)trihydroxyphenyl) is replaced with a lipophilic moiety (e.g., OC(O)CH₂(CH₂)₅CH₃, or OC(O)CH₂(CH₂)₇CH₃).

As used herein, the term “modified catechin” generally refers to a molecule having a catechin scaffold, wherein the catechin scaffold may optionally be substituted with one or more substituents (e.g., a hydroxyl group), and wherein the catechin scaffold includes at least one substituent of the formula OC(O)R, OC(S)R, OC(NH)R, OR, or R, wherein R is optionally substituted alkyl, alkenyl, alkynyl, alkaryl, or aryl.

The term “alkyl” as used herein includes all saturated hydrocarbon groups in a straight, branched, or cyclic configuration (also referred to as cycloalkyl, see below), and particularly contemplated alkyl groups include lower alkyl groups (i e., those having six or less carbon atoms). Exemplary alkyl groups are methyl, ethyl, propyl, isopropyl, butyl, sec-butyl, tertiary butyl, pentyl, isopentyl, hexyl, isohexyl, etc. The term “alkenyl” as used herein refers an alkyl as defined above having at least one double bond. Thus, particularly contemplated alkenyl groups include straight, branched, or cyclic alkene groups having two to six carbon atoms (e.g., ethenyl, propenyl, butenyl, pentenyl, etc.). Similarly, the term “alkynyl” as used herein refers an alkyl or alkenyl as defined above having at least one triple bond, and especially contemplated alkynyls include straight, branched, or cyclic alkynes having two to six total carbon atoms (e.g., ethynyl, propynyl, butynyl, pentynyl, etc.).

The term “cycloalkyl” as used herein refers to a cyclic alkyl (i.e., in which a chain of car-bon atoms of a hydrocarbon forms a ring), preferably including three to eight carbon atoms. Thus, exemplary cyclooalkanes include cyclopropyl, cyclobutyl, cyclopentyl, cyclohexyl, cycloheptyl, and cyclooctyl. Contemplated cycloalkyls may further include one or more double and/or triple bonds, which may be conjugated. The term “aryl” as used herein refers to an aromatic carbon atom-containing ring, which may further include one or more non-carbon atoms. Thus, contemplated aryl groups include cycloalkenes (e.g., phenyl, naphthyl, etc.) and pyridyl.

The term “substituted” as used herein refers to a replacement of an atom or chemical group (e.g., H, NH₂, or OH) with a functional group, and particularly contemplated functional groups include nucleophilic groups (e.g., —NH₂, —OH, —SH, —NC, etc.), electrophilic groups (e.g., C(O)OR, C(X)OH, etc.), polar groups (e.g., —OH, C(O)Cl, etc.), non-polar groups (e.g., aryl, alkyl, alkenyl, alkynyl, etc.), ionic groups (e.g., —NH₃⁺), and halogens (e.g., —F, —Cl), and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties. The term “functional group” and “substituent” are used interchangeably herein and refer to a groups including nucleophilic groups (e.g., —NH₂, —OH, —SH, —NC, —CN etc.), electrophilic groups (e.g., C(O)OR, C(X)OH, C(Halogen)OR, etc.), polar groups (e.g., —OH), non-polar groups (e.g., aryl, alkyl, alkenyl, alkynyl, etc.), ionic groups (e.g., —NH₃⁺), and halogens.

As also used herein, the term “reduce bacterial growth” refers to any mode of reduction in number of bacteria, and/or any reduction in the rate of bacterial cell division. Such reduction may be precipitated by one or more manners, and specifically contemplated manners include cell membrane damage, cytotoxic effects, reduction in cell wall synthesis, and/or reduction in nucleic acid synthesis. The term “damages a bacterial membrane” as used herein refers to any change in a bacterial cell membrane that reduces viability, cell division, and/or structural integrity of the cell membrane. Such reduction may involve several mechanisms, including perturbation of lipid bilayer structure, pore formation, disruption of membrane gradients, etc.

Contemplated Compounds

Based on the discovery of the inventors that a relatively wide range of modifications may be made to produce antibacterially active modified catechins, it is generally contemplated that suitable compounds according to the inventive subject matter will have a general structure of Formula 1

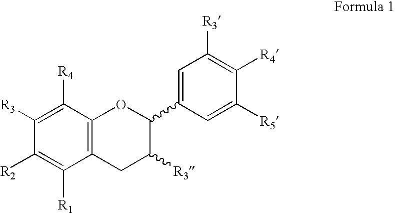

wherein R₁, R₂, R₃, R₄, R₃′, R₄′, and R₅′ are independently H, OH, or M, wherein R₃″ is H, OH, an optionally substituted phenyl, or M, with the proviso that at least one of R₁, R₂, R₃, R₄, R₃′, R₄, R₅′, and R₃″ is M; and wherein M is OC(O)R, OC(S)R, OC(NH)R, OR, or R, wherein R is optionally substituted allyl, alkenyl, alkynyl, alkaryl, or aryl; It is further contemplated that M may also include membrane lipids or portions thereof, including a cholinyl or glyceryl moiety (preferably covalently coupled to an acyl, alkyl, alkenyl, alkynyl, or aryl), or a steroid moiety (e.g., cholesterol and its variations that occur in biological membrane).

In one particularly preferred aspect, contemplated compounds will have a structure according to Formula 2 or Formula 4 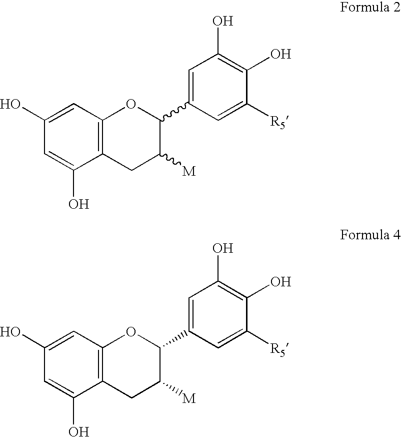
wherein R₅′ is H or OH, and wherein M is OC(O)R, and even more preferably OC(O)CH₂(CH₂)₅CH₃, or OC(O)CH₂(CH,)₇CH₃.

It should further be recognized that contemplated compounds typically exist in various stereoisomeric configurations (e.g., 2-R,S and/or 3-R,S), and it should be appreciated that all isomeric forms (including enantiomeric isoforms, diasteriomeric isoforms, tautomeric isoforms, etc.) are expressly included herein. Moreover, especially where contemplated compounds are synthesized entirely in a lab, one or more isoforms may be separated from another isoform to yield an optically pure single isomeric form, or a defined mixture of two or more isoforms. On the other hand, modified catechins may be prepared from crude or refined extracts from a plant source, and the so obtained catechins may be isomerically pure at least to some extent (which will typically depend on the particular plant material and isolation process).

Furthermore, where appropriate, contemplated compounds may also be prepared as salts, and especially suitable salts include those formed with an organic or inorganic acid/base to provide a pharmaceutically acceptable salt (e.g., HCl salt, mesylate, etc). While not especially preferred, it should be recognized that contemplated compounds may also be polymerized to at least some degree.

Contemplated Uses

Based on the discovery of the inventors that contemplated compounds exhibit significant antibacterial activity, and on the further observation that contemplated compounds may damage bacterial lipid bilayer membranes (infra), the inventors generally contemplate that that modified catechins may be employed as antimicrobial agent in a variety of products.

For example, where additional beneficial activities (e.g., anti-oxidant) of contemplated compounds are desired, modified catechins may be added to a cosmetic formulation as a preservative and/or a dermatological desirable compound. Therefore, and depending on the particular compound, application, and formulation, modified catechins may preferably be included in a range of between about 0.001 wt % to about 5 wt % (and even more). With respect to the type of cosmetic formulation, it should be recognized that all known cosmetic formulations are considered suitable, and especially include facial creams and lotions, moisturizing creams and lotions, lipstick, etc. Therefore, the composition of the specific cosmetic formulation may vary significantly, and it is generally contemplated that all known cosmetic formulations are considered suitable for use herein. Exemplary guidance on how to prepare suitable cosmetic formulations can be found in “Cosmetic and Toiletry Formulations”, Volume 8, by Ernest W. Flick; Noyes Publications; 2nd edition (Jan. 15, 2000) (ISBN: 0815514549), which is incorporated by reference herein.

In another example, contemplated compounds may be employed as antimicrobial agent in a pharmaceutical composition, wherein it is generally preferred that the modified catechin is present at a concentration effective to reduce bacterial growth in a body compartment (e.g., skin, open wound, eye, mucous membrane, infected organ, blood) when administered to the body compartment. For example, contemplated compounds may be added as a preservative to a liquid, solid, or other form of a pharmacological agent, and it is generally contemplated that in such function, the amount of modified catechins will preferably be in the range of between about 0.01 wt % to about 1.0 wt %. Where the modified catechin is employed as an antioxidant, suitable concentrations of the modified catechin in the pharmaceutical composition will generally be in a somewhat higher range, including a range of between about 0.1 wt % to about 5.0 wt %.

On particularly preferred embodiment is a topically applied pharmaceutical composition (e.g., spray, ointment, lotion, or cream) that includes one or more of contemplated compounds as a topical antimicrobial agent for skin and/or wound infections. Contemplated pharmaceutical compositions may be particularly advantageous where the infection is caused by a microorganism that is otherwise resistant to treatment with one or more antibiotic drugs. For example, it is contemplated that the resistant bacterium is Staphylococcus aureus, which may be resistant to methicillin (and/or other beta-lactam antibiotics, cephalosporins, and/or vancomycin). Depending on the specific formulation (e.g., spray, ointment, lotion, or cream), the particular composition of the pharmaceutical composition may vary considerably. Further particularly contemplated microorganisms that may be exposed to contemplated compounds via a cosmetic and/or pharmaceutical composition include Streptococcus pyogenes, Streptococcus agalactiae, Propionobacterium acne, or Listeria monocytogenes. Exemplary guidance for preparation of contemplated formulations can be found in “Dermatological and Transdermal Formulations”, (Drugs and the Pharmaceutical Sciences, Vol. 119), by Kenneth A. Walters, Marcel Dekker; (February 2002) (ISBN: 0824798899). With respect to the concentration of contemplated compounds it is generally preferred that modified catechins will be present in an amount of at least 0.001 wt %, more preferably of at least 0.01-0.1 wt %, and more preferably of at least 0.01-5.0 wt %.

In a still further example, contemplated compounds may also be included into various cleaning formulations, and especially contemplated cleaning formulations include household cleaning fluids (e.g., liquid dish soap, surface disinfectants, etc) and personal grooming items (e.g., toothpaste, mouthwash, shower gel, deodorant, etc.). Once more, the general composition of such cleaning formulations is well known in the art, and preferred quantities of contemplated compounds in such products will generally be identical with quantities provided for the pharmaceutical compositions provided above.

In yet another aspect of the inventive subject matter, it should be recognized that the antibacterial activity of contemplated compounds is not limited to multi-drug resistant strains of S. aureus. In fact, the inventors contemplated that all types of bacteria can be treated with contemplated compounds and compositions. However, it is generally preferred that the bacteria particularly include gram-positive bacteria. Moreover, contemplated compositions may also exhibit to at least some degree antifungal activity.

Therefore, viewed from a more general perspective, it should be recognized that a method of reducing growth of a bacterium may include a step in which bacteria are contacted with a modified catechin at a dosage effective to reduce growth of the bacteria. The term “contacting a bacterium” with a modified catechin as used herein means that the bacterium is exposed to the modified catechin in a manner that allows molecular interaction between the modified catechin and a component of the bacterium (e.g., cell membrane, periplasmic enzyme, cell wall, etc.). Therefore, where the bacteria reside on the surface of a skin or wound, the step of contacting may include directly applying a cream, lotion, spray, or other topical formulation to the skin or wound. On the other hand, where the bacteria reside in the blood or an organism, the step of contacting may include injection (e.g., i.v., or i.m.) of contemplated compounds to the blood stream.

Consequently, a method of marketing may include a step in which a product is provided that includes a modified catechin according to the inventive subject matter. In another step, it is advertised that the product reduces bacterial growth. Advertising may include numerous manners of disseminating information, and especially preferred manners include providing printed information (e.g., package insert, package labeling, flyer, advertisement in a magazine, etc.) suggesting or describing reduction of bacterial growth, or providing televised information (e.g., TV commercial, or TV, infomercial) suggesting or describing reduction of bacterial growth.

### EXAMPLES

### Methods

Reagents and bacterial strains: 3-O-(—)-epicatechingallate and (+)-catechin were provided by the Tokyo Food Techno Co., Tokyo, Japan. Octanoic acid and oxacillin were purchased from Sigma (Poole, United Kingdom). The acyl-(+)-catechin derivatives and octanoyl-(-)-epicatechin were synthesised as outlined below. S. aureus BB568 (COL-type strain that carries mecA and pT181) and BB551 (methicillin-sensitive) were provided by Professor B. Berger-Baechi. EMRSA-15 and EMRSA-16 were clinical isolates from the Royal Free Hospital, London. Strains of S. aureus can be considered resistant to methicillin in which growth occurs in the presence of 8 micrograin/ml methicillin (National Committee for Clinical Laboratory Standards, 1990—Methods for dilution antimicrobial susceptibility tests for bacteria that grow aerobically (second edition). Document M7-A2. NCCLS, Villanova, Pa., U.S.A.).

Minimum inhibitory concentration: MIC testing was performed in 96-well microtitre trays with an inoculum of about 10⁴ CFU in 100 microliter of Mueller-Hinton broth (Oxoid, Basingstoke, United Kingdom) supplemented with 2% NaCI. MIC values were obtained after incubation at 35° C. for 24 h. S. aureus ATCC29213 was used as the standard.

Effect on bacterial growth: EMRSA-16 was grown overnight in Mueller-Hinton broth at 37° C. The overnight culture as diluted 1:400 into 50 ml volumes of pre-warmed (37° C.) Mueller-Hinton broth containing various concentrations of contemplated compounds. The control flask contained ethanol (1 vol %). The flasks were incubated at 37° C. with aeration (200 rpm). At two-hour intervals samples were withdrawn from the flasks, serially diluted in 0.1M phosphate-buffered saline (pH 7.4) solutions, and plated onto nutrient agar (Oxoid). The number of colonies was recorded at 24 h incubation at 37° C. and expressed as the number of CFU/ml.

Bacterial membrane damage: EMRSA-16 was grown overnight in Mueller-Hinton broth at 37° C. The overnight culture was deluted 1:40 into fresh pre-warmed Mueller-Hinton broth and the diluted culture incubated at 37° C., with aeration (200 rpm), until the optical density at 600 nm (OD₆₀₀) reached 0.7-0.8. The cells were recovered by centrifugation (10.000×g for 10 min), washed once with filtered sterilized water, and resuspended to 1:10 the original volume in filter-sterilized water. The culture was further diluted 1:20 into water containing ethanol (1 vol %; the solvent was used to dissolve the compounds) or water containing the catechin. The cells were exposed to the compounds for 10 min (at room temperature and gentle shaking), after which a sample was removed for CFU determination and the remainder of the cells were recovered by centrifugation (10.000×g for 10 min). The cell pellet was washed once with water and then resuspended to an OD₆₇₀ of 0.15.

Damage to the bacterial cytoplasmic membrane was determined with the reagents (SYTO 9 and propidium iodide) contained in the BacLight kit from Molecular Probes Europe BV (Leiden, The Netherlands). An equal mixture (4.5 microliter each) of SYTO 9 dye and propidium iodide was added to 3 ml of sample in a cuvette and the sample mixed by inversion of the cuvette three times. The sample was maintained in the dark for 15 min and the fluorescence of the two dyes was determined with a spectrofluorometer (Jacso FP-750). Both dyes were excited with a wavelength of 485 nm and the emission of SYTO 9 was read at 530 nm (Em1) and propidium iodide was read at 645 nm (Em2). The ratio of SYTO 9 to propidium iodide emissions (R=Em1/Em2) was expressed as a percentage of the control (BacLight value=[Rsample/Rcontrol]×100). The sample removed for CFU determination was serially diluted in 0.1M phosphate-buffered saline (pH 7.4) then plated onto nutrient agar. The number of colonies on the plates was recorded after 24 h incubation at 37° C. and the results expressed as a Log10 decrease in CFU/ml compared to the control sample.

Erythrocyte haemolysis: Erythrocytes from defibrinated Horse blood (Oxoid) were collected by centrifugation (6,000×g, 3 min) and washed three to four times in 10 mM Tris-HCl (pH 7.4) containing 0.9% NaCI. The erythrocytes were resuspended to 1% in the wash buffer and 200 microliter of cells was added to 1300 microliter of buffer containing the test compound. The sample was mixed gently for 10 min at room temperature and the intact erythrocytes were removed by centrifugation (6,000×g, 3 min). Haemolysis was evaluated by measuring the absorbance of the supernatant at 540 nm. Cells were added to buffer containing 0.5% NH₄OH to give an indication of 100% lysis. The results were expressed as a percentage of absorbance reading for 100% lysis. Buffer containing only washed erythrocytes was used to assess the extent of lysis in the absence of the test compound.

Electron microscopy: S. aureus BB551 was grown overnight at 37° C. in Mueller-Hinton broth in the absence and presence of either epicatechin-(−)-gallate or octanoyl-(+)-catechin. The cells were recovered by centrifugation and washed once in 0.1M phosphate-buffered saline, pH 7.4. Cells were fixed in 1.5% glutaraldehyde for at least 2 h at room temperature, treated with osmium tetroxide and embedded in epoxy resin. Sectioning and staining with uranyl acetate was followed by Reynolds' lead citrate. The ultrathin sections were viewed and photographed using a Philips 201 transmission electron microscope.

### Results

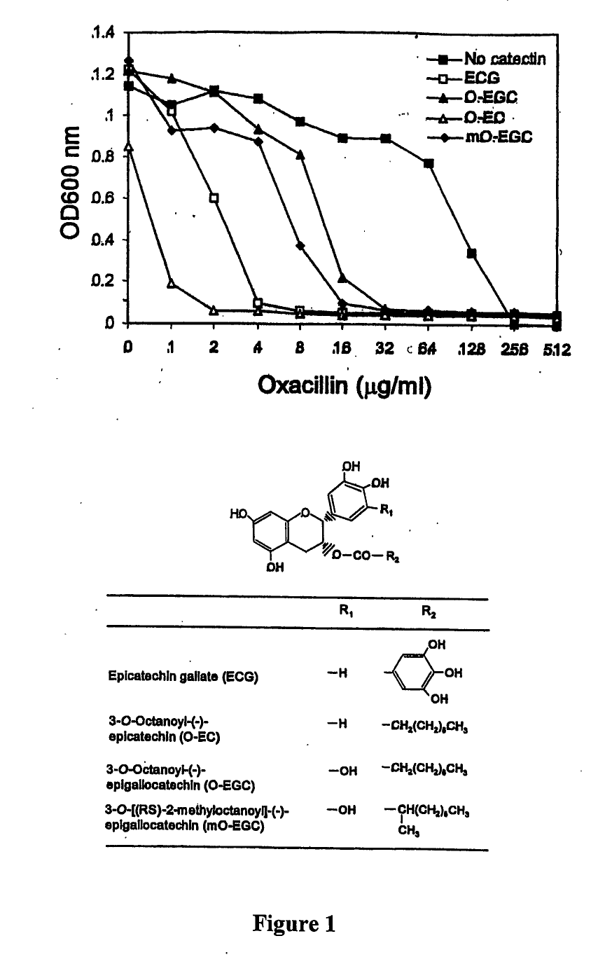

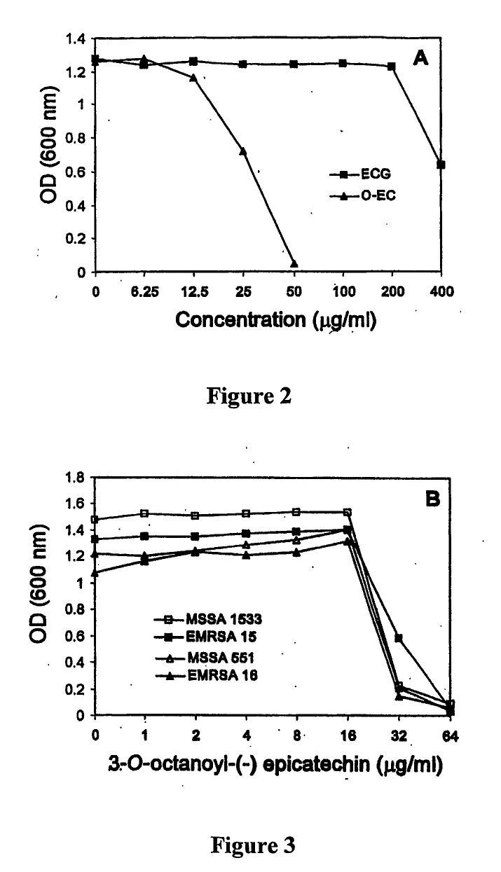

Bactericidal activities: The effect of various modified catechins against EMRSA-15 was tested at a predetermined dose of selected modified catechins on a methicillin resistant strain of S. aureus in the presence of rising doses of Oxacillin as depicted in FIG. 1. Clearly, 3-O-octanoyl-(−)-epicatechin (O-EC) exhibited significant antimicrobial effect at even zero concentration of oxacillin. FIG. 2 shows the dose-dependent antimicrobial effect of O-EC on a methicillin resistant strain of S. aureus as compared to epicatechingallate in the absence of an antibiotic. Once more, O-EC demonstrated superior antibacterial effect, even at relatively low dosages. To further investigate the antimicrobial effect on other methicillin-resistant strains, O-EC was added to various S. aureus cultures (MSSA 1533, MSSA 511, EMRSA-15, and EMRSA-16). Remarkably, all of the strains exhibited similar susceptibility towards O-EC at about same concentrations as depicted in FIG. 3.

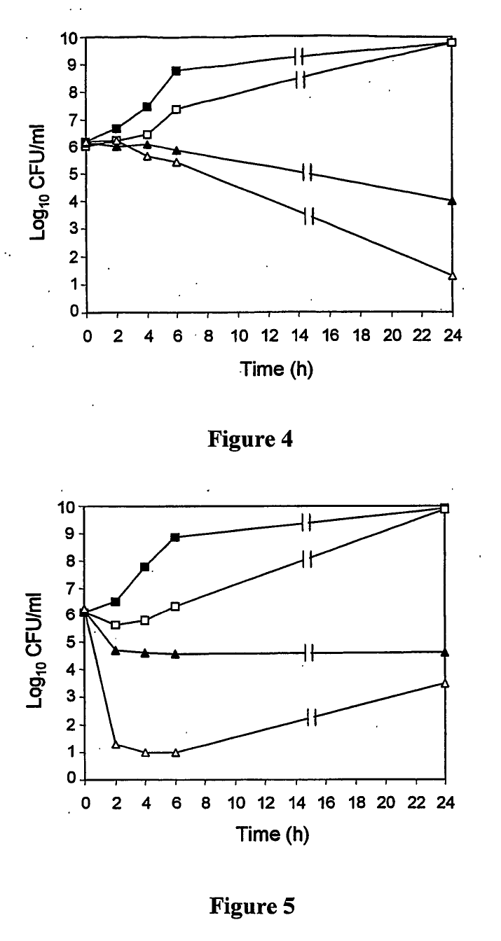

When incubated with ECG, the inventors observed that ECG did not give rise to a large reduction in viable cell numbers over the first two hour period, even at 8×SIC. Instead, a slight reduction in cell numbers (0.3 and 0.85 Log10 reduction for 512 and 1024 microgram/ml, respectively) was observed over six hours. The number of viable cells decreased further over the 24 h period giving rise to a 5 Log10 reduction in CFU/ml when grown in the presence of ECG at 1024 microgram/ml. An exemplary growth pattern is depicted in FIG. 4.

In contrast, a distinct effect was observed for octanoyl-(+)-catechin on the growth of EMRSA-16 as shown in FIG. 5: At an octanoyl(+)-catechin concentration of 32 microgram/ml, there was an initial 1.6 Log10 reduction in the number of viable cells and growth was inhibited over the 24 h period investigated. At 64 microgram/ml the-compound was bactericidal giving rise to a 5 Log10 reduction in viable cell numbers after 2 h incubation. Slight re-growth was observed after 24 h. Cells that grew after 24 h were tested for susceptibility to octanoyl-(+)-catechin; no decrease in susceptibility was observed (data not shown).

Minimum inhibitory concentrations: (+)-Catechin had a MIC>256 microgram/ml for the three strains tested. ECg had at least 4-fold greater direct antistaphylococcal activity than (+)-catechin, although the activity was still poor (64-128 microgram/ml). Introduction of acyl chains to (+)-catechin generally enhanced the antistaphylococcal activity of the molecule. 3-O-acyl-(+)-catechins where chain lengths of C4, C6, C16 and C18 had MICs greater or equal than 32 microgram/ml for S. aureus BB568. Compounds with chain lengths of C8, C10, C12 and C14 had consistently lower MICs (16 microgram/ml) when tested against S. aureus BB568 and EMRSA-16 but chain lengths of C12 and C14 were less effective against EMRSA-15 (greater or equal than 32 microgram/ml). 3-O-octanoyl-(−)-epicatechin had similar activity to 3-O-octanoyl-(+)-catechin, and octanoic acid had no direct activity against S. aureus. Of the compounds tested, only epicatechin gallate was able to significantly reduce the oxacillin MIC (256 to less than 1 microgram/ml. None of the acyl catechin derivatives or octanoic acid (tested at 0.25×MIC) had the capacity to reduce the oxacillin MIC greater than two-fold.

| | MINIMUM INHIBITORY CONCENTRATION (MIC) | | | | | |
|---|---|---|---|---|---|---|
| | IN MICROGRAM/ML | | | | | |
| | BB568 | | EMRSA-15 | | EMRSA-16 | |
| COMPOUND | Catechin | Oxacillin | Catechin | Oxacillin | Catechin | Oxacillin |
| Oxacillin | | 256 | | 32 | | 512 |
| 3-O-butyroyl-(+)- | >64 | 128 | >64 | 32 | >64 | 256 |
| catechin | | | | | | |
| 3-O-hexanoyl-(+)- | 64 | 128 | 64 | 16 | 64 | 256 |
| catechin | | | | | | |
| 3-O-octanoyl-(+)- | 16 | 256 | 16 | 32 | 16 | 256 |
| catechin | | | | | | |
| 3-O-decanoyl-(+)- | 16 | 128 | 16 | 16 | 16 | 256 |
| catechin | | | | | | |
| 3-O-dodecanoyl-(+)- | 16 | 128 | >16 | 32 | 16 | 256 |
| catechin | | | | | | |
| 3-O-myristoyl-(+)- | 16 | 128 | >32 | 32 | 16 | 512 |
| catechin | | | | | | |
| 3-O-palmitoyl-(+)- | 32 | 256 | >32 | 32 | 16 | 512 |
| catechin | | | | | | |
| 3-O-staeoryl-(+)- | 32 | 256 | >32 | 32 | >32 | 512 |
| catechin | | | | | | |
| (+)-catechin | >256 | 256 | >256 | 32 | >256 | 512 |
| (−)-epicatechingallate | 128 | <1 | 128 | 1 | 128 | 1 |
| 3-O-octanoyl-(−)- | 32 | 256 | 32 | 32 | 16 | 256 |
| epicatechin | | | | | | |
| Octanoic acid | 1024 | 256 | 1024 | 32 | 1024 | 512 |

Staphylococcal membrane damage: Damage to the staphylococcal cytoplasmic membrane was assessed by use of the BacLight kit (Molecular Probes Inc.). The kit makes use of two nucleic acid stains, SYTO-9 and propidium iodide, with different spectral properties and abilities to penetrate intact bacterial membranes. SYTO-9 penetrates both intact and damaged membranes while propidium iodide only penetrates damaged membranes. Cells with intact membranes stain fluorescent green while cells with damaged membranes stain fluorescent red. The ratios of green to red fluorescence, for EMRSA-16 exposed to test compounds, are expressed as a percentage of the control and are given in the table below. Octanoyl-(+)-catechin when tested at the MIC resulted in significant membrane damage (98% increase in permeability when compared to the untreated control) and resulted in a 2.6 Log10 reduction in the number of viable cells. At an octanoyl-(+)-catechin concentration twice the MIC a greater than 7 Log10 reduction in the number of viable cells was observed despite the short exposure time of 10 min. Epicatechin gallate when tested at 4× and 8×MIC only resulted in moderate membrane permeability (48% and 64%, respectively) and there was little effect on cell viability. Octanoic acid only gave rise to significant membrane damage at very high concentrations (>1024 microgram/ml).

Hemoglobin released from horse blood erythrocytes after exposure to the compounds for 10 min was used to assess the effect of the compounds on eukaryotic membranes. With this assay octanoyl-(+)-catechin was shown to be significantly hemolytic at the MIC (24% hemolysis) and above (100%) as indicated in the table below. ECg did not give rise to hemolysis at 4× MIC but hemolysis was observed at 8× MIC (21%). Octanoic acid at 2× MIC gave rise to complete hemolysis.

| | Membrane Effect | | | |
|---|---|---|---|---|
| | Concentration | % Control | Delta Log10 | % |
| Compound | tested (mcg/ml) | (BacLight) | (CFU/ml) | Hemolysis |
| Ocanoyl-(+)- | 4 | 75 | −0.1 | 4 |
| catechin | 8 | 24 | 0.2 | 5 |
| | 16 | 2 | 2.6 | 24 |
| | 32 | 2 | >7.0 | 100 |
| | 64 | 2 | >7.0 | 100 |
| Octanoic acid | 16 | 74 | −0.1 | 3 |
| | 32 | 75 | 0.1 | 4 |
| | 64 | 2 | >7.0 | 100 |
| Epicatechin-(−)- | 512 | 48 | 0.0 | 6 |
| gallate | 1024 | 64 | 0.1 | 21 |
| Untreated | 0 | 100 | 0.0 | 4 |
| Control | | | | |

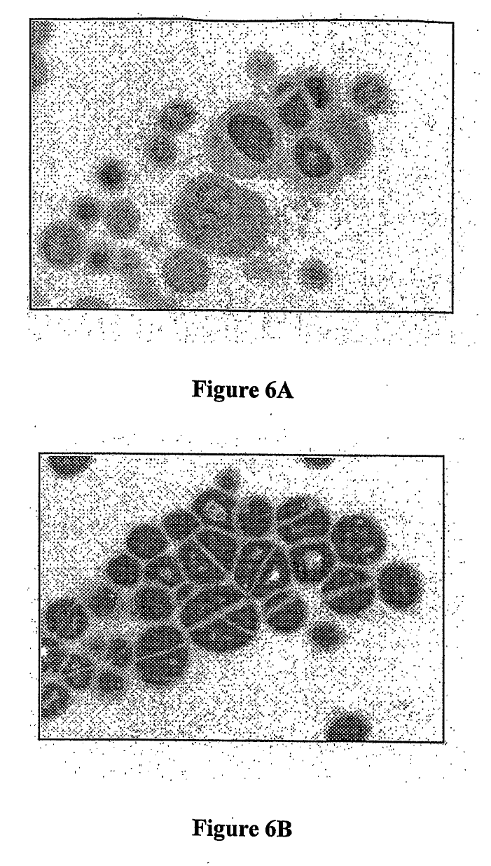

Effect on cell wall morphology: Growth of S. aureus BB551 in the presence of ECg gave rise pseudomulticellular aggregates with increased cell wall thickening (FIG. 6A). The same strain grown in the presence of 3-O-octanoyl-(−)-epicatechin also gave rise to pseudomulticellular aggregates but no cell wall thickening was observed. Aberrant septa formation was also noted (FIG. 6B).

Synthesis of contemplated compounds: It is generally contemplated that a person of ordinary skill in the art will readily be able to devise a synthetic strategy for contemplated compounds. Nevertheless, exemplary references are provided below for numerous of contemplated compounds, and it should be recognized that such synthetic procedures may be modified to arrive at the particular molecule not specifically disclosed in those references. Lambusta et al., in Synthesis 1993, p. 1155-1158 reported the preparation of [(+)-3-0-ACETYLCATECHIN] by alcoholysis of peracetylated (+)-catechin in the presence of Pseudomonas cepacia lipase. EP 0618203 reports catechins acylated at position C-3, prepared by esterifications of free catechin catalysed by Streptomyces rachei or Aspergillus niger carboxylesterase. Nicolosi et al. describe in WO 99/66062 a procedure to obtain 3-monoesters of a flavonoid as the only reaction product by carrying out the alcoholysis of a peracylated flavonoid in organic solvent in the presence of Mucor miehei lipase. Kozikowski et al report in J. Org. Chem. Aug. 25, 2000;65(17):5371-81 synthesis of 3-O-alkylated flavonoids. The C-3 hydroxyl group can be removed via modified Barton deoxygenation using hypophosphorous acid as the reducing agent. C—C bond formation may in 3-position may be achieved via alkylMgBr reaction, or via Heck, Suzuki, or Stille reaction.

### 3-O-butyryl-(+)-catechin

(+)-catechin (1.00 g, 3.44 mmol) and butyryl chloride (0.179 ml, 1.68 mmol) were dissolved in tetrahydrofuran (10 mL) containing trifluoroacetic acid (0.270 ml, 3.55 mmol), and the solution was stirred for 17 hrs under an Ar gas at room temperature. The reaction mixture was diluted with CHCl₃—MeOH (3:1) and washed five times with water. The organic layer was concentrated in vacuo to give a residue. Purification by the preparative HPLC using a GS-320 column (21.5 mm ID×500 mm) with MeOH as an eluent., followed by freeze-drying, yielded the desired 3-O-butyryl-(+)-catechin 85 mg as white powder (14.0% yield). [α]²⁰_{D}+7.8° (EtOH, c=0.5); IR (KBr) 3707, 2607, 2326, 1697, 1504, 1454, 1140, 1013, 833, 781, 419 cm⁻¹; ¹H NMRδ: 0.79 (3H, t, J=7.4 Hz, —COCH₂CH₂CH₃), 1.45-1.53 (2H, m, —COCH₂CH₂CH₃), 2.13-2.19 (2H, m, —COCH₂CH₂CH₃), 2.58-2.62 (1H, m, H-4), 2.78-2.82 (1H, m, H-4), 5.17-5.21 (1H, m, H-3), 5.88 (1H, s, H-6 or H-8), 5.93 (1H, s, H-8 or H-6), 6.65-6.68 (1H, m, H-2′), 6.72 (1H, d, J=8.0 Hz, H-3′), 6.78 (1H, s, H-6′); HR-FABMS m/z: 361.1285 ([M+H]⁺, Calcd for C₁₉H₂₁O₇: 361.1287).

### 3-O-hexanoyl-(+)-catechin

(+)-catechin (1.01 g, 3.48 mmol) and hexanoyl chloride (0.242 ml, 1.80 mmol) were dissolved in tetrahydrofuran (10 mL) containing trifluoroacetic acid (0.270 ml, 3.55 mmol). The solution was treated in the same way as for Example 1, yielding 3-O-hexanoyl-(+)-catechin 113 mg as white powder (16.8 % yield). [α]²⁰_{D}+4.7° (EtOH, c=0.5); IR (KBr) 3732, 2927, 2358, 1867, 1715, 1605, 1520, 1456, 1362, 1252, 1140, 1015, 827, 667, 419 cm⁻¹; ¹H NMRδ: 0.83 (3H, t, J=7.4 Hz, —COCH₂CH₂(CH₂)₂CH₃), 1.10-1.23 (4H, m, —COCH₂CH₂(CH₂)₂CH₃), 1.41-1.45 (2H, m, —COCH₂—CH₂(CH₂)₂CH₃), 2.18 (2H, t, J=7.0 Hz, —COCH₂CH₂(CH₂)₂CH₃), 2.58 (1H, dd, J=6.8, 16.0 Hz, H-4), 2.79-2.83 (1H, m, H-4), 5.18 (1H, d, J=5.6 Hz, H-3), 5.87 (1H, s, H-6 or H-8), 5.93 (1H, s, H-8 or H-6), 6.63-6.66 (1H, m, H-2′), 6.71 (1H, d, J=7.6 Hz, H-3′), 6.78 (1H, s, H-6′); HR-FABMS m/z: 389.1578 ([M+H]⁺, Calcd for C₂₁H₂₅O₇: 389.1600).

### 3-O-octanoyl-(+)-catechin

(+)-catechin (1.02 g, 3.51 mmol), octanoyl chloride (0.290 ml, 1.70 mmol) and trifluoroacetic acid (0.270 ml, 3.55 mmol) were dissolved in tetrahydrofuran (10 mL). The solution was treated in the same way as for Example 1, yielding 3-O-octanoyl-(+)-catechin 214 mg as white powder (16.7 % yield). [α]²⁰_{D}+5.2° (EtOH, c=0.4); IR (KBr) 3310, 2928, 2856, 2359, 1734, 1622, 1607, 1528, 1518, 1475, 1389, 1300, 1254, 1150, 1057, 1028, 964, 829, 731, 669 cm⁻¹; ¹H NMRδ: 0.89 (3H, t, J=6.7 Hz, —COCH₂CH₂(CH₂)₄CH₃), 1.12-1.33 (8H, m, —COCH₂CH₂(CH₂)₄CH₃), 1.39-1.49 (2H, m, —COCH₂CH₂(CH₂)₄CH₃), 2.20 (2H, t, J=7.2 Hz, —COCH₂CH₂(CH₂)₄CH₃), 2.59 (1H, dd, J=7.2, 16.2 Hz, H-4), 2.81 (1H, dd, J=5.6, 16.2 Hz, H-4), 5.16-5.23 (1H, m, H-3), 5.88 (1H, d, J=2.4 Hz, H-6 or H-8), 5.94 (1H, d, J=2.2 Hz, H-8 or H-6), 6.67 (1H, dd, J=1.9, 8.2 Hz, H-2′), 6.73 (1H, d, J=8.2 Hz, H-3′), 6.79 (1H, d, J=1.9 Hz, H-6′); HR-FABMS m/z: 417.1906 ([M+H]⁺, Calcd for C₂₃H₂₉O₇: 417.1914).

### 3-O-decanoyl-(+)-catechin

(+)-catechin (1.01 g, 3.48 mmol) and decanoyl chloride (0.362 ml, 1.90 mmol) were dissolved in tetrahydrofuran (10 mL) containing trifluoroacetic acid (0.270 ml, 3.55 mmol). The solution was treated in the same way as for Example 1, yielding 3-O-decanoyl-(+)-catechin 124 mg as white powder (16.0 % yield). [α]²⁰_{D}+13.4° (EtOH, c=0.4); IR (KBr) 3352, 2922, 2852, 1711, 1632, 1518, 1468, 1359, 1245, 1140, 1063, 818, 419 cm⁻¹; ¹H NMRδ: 0.07 (3H, t, J=6.8 Hz, —COCH₂CH₂(CH₂)₆CH₂), 0.32-0.49 (12H, m, —COCH₂CH₂(CH₂)₆CH₃), 0.58-0.65 (2H, m, —COCH₂CH₂(CH₂)₆CH₃), 1.37 (2H, t, J=7.0 Hz, —COCH₂CH₂(CH₂)₆CH₃), 1.76 (1H, dd, J=7.0, 16.6 Hz, H-4), 1.98 (1H, dd, J=5.4, 16.6 Hz, H-4), 4.35-4.39 (1H, m, H-3), 5.06 (1H, s, H-6 or H-8), 5.11 (1H, s, H-8 or H-6), 5.82-5.86 (1H, m, H-2′), 5.90 (1H, d, J=7.6 Hz, H-3′), 5.96 (1H, s, H-6′); HR-FABMS m/z: 445.2260 ([M+H]⁺, Calcd for C₂₅H₃₃O₇: 445.2227).

### 3-O-dodecanoyl-(+)-catechin

(+)-catechin (1.00 g, 3.44 mmol) and dodecanoyl chloride (0.396 ml, 1.81 mmol) were dissolved in tetrahydrofuran (10 mL) containing trifluoroacetic acid (0.270 ml, 3.55 mmol). The solution was treated in the same way as for Example 1, yielding 3-O-dodecanoyl-(+)-catechin 118 mg as white powder (14.5 % yield). [α]²⁰_{D}+1.50 (EtOH, c=0.5); IR 3609, 3560, 3302, 2924, 2328, 1713, 1659, 1518, 1452, 1286, 1140, 1016, 665, 517 cm⁻¹; ¹H NMRδ: 1.04 (3H, t, J=6.6 Hz, —COCH₂CH₂(CH₂)₈CH₃), 1.29-1.52 (16H, m, —COCH₂CH₂(CH₂)₈CH₃), 1.57-1.60 (2H, m, —COCH₂CH₂(CH₂)₈CH₃), 2.34 (2H, t, J=7.4 Hz, —COCH₂CH₂(CH₂)₈CH₃), 2.74 (1H, dd, J=7.0, 16.2 Hz, H-4), 2.95 (1H, dd, J=5.0, 16.2 Hz, H-4), 5.33-5.35 (1H, m, H-3), 6.03 (1H, s, H-6 or H-8), 6.08 (1H, s, H-8 or H-6), 6.80-6.83 (1H, m, H-2′), 6.87 (1H, d, J=8.0 Hz, H-3′), 6.94 (1H, s, H-6′); HR-FABMS m/z: 473.2548 ([M+H]⁺, Calcd for C₂₇H₃₇O₇: 473.2540).

### 3-O-myristoyl-(+)-catechin

(+)-catechin (0.99 g, 3.41 mmol) and myristoyl chloride (0.464 ml, 1.88 mmol) were dissolved in tetrahydrofuran (10 mL) containing trifluoroacetic acid (0.270 ml, 3.55 mmol). The solution was treated in the same way as for Example 1, yielding 3-O-myristoyl-(+)-catechin 73 mg as white powder (8.6 % yield). [α]²⁰_{D}+1.0° (EtOH, c=0.7), IR (KBr) 3612, 2922, 2853, 2357, 1715, 1651, 1520, 1456, 1362, 1142, 1061, 816, 419 cm⁻¹; ¹H NMRδ: 0.08 (3H, t, J=6.6 Hz, —COCH₂CH₂(CH₂)₁₀CH₃), 0.43-0.53 (20H, m, —COCH₂CH₂(CH₂)₁₀CH₃), 0.62-0.65 (2H, m, —COCH₂CH₂(CH₂)₁₀CH₃), 1.38 (2H, t, J=7.4 Hz, —COCH₂CH₂(CH₂)₁₀CH₃), 1.79 (1H, dd, J=7.4, 16.0 Hz, H-4), 2.00 (1H, dd, J=5.2, 16.0 Hz, H-4), 4.38-4.41 (1H, m, H-3), 5.01 (1H, s, H-6 or H-8), 5.13 (1H, s, H-8 or H-6), 5.84-5.88 (1H, m, H-2′), 5.92 (1H, d, J=8.0 Hz, H-3′), 5.98 (1H, s, H-6′); HR-FABMS m/z: 501.2861 ([M+H]⁺, Calcd for C₂₉H₄₁O₇: 501.2853).

### 3-O-palmitoyl-(+)-catechin

(+)-catechin (1.00 g, 3.44 mmol) and palmitoyl chloride (0.523 ml ,1.90) were dissolved in tetrahydrofuran (10 mL) containing trifluoroacetic acid (0.270 ml, 3.55 mmol). The solution was treated in the same way as for Example 1, yielding 3-O-palmitoyl-(+)-catechin 70 mg as white powder (7.7% yield). [α]²⁰_{D}+16.4° (EtOH, c=0.5); IR (KBr) 3736, 2918, 2851, 2498, 1747, 1606, 1521, 1474, 1362, 1254, 1144, 1057, 814, 419 cm⁻¹; ¹H NMRδ: 0.08 (3H, t, J=6.8 Hz, —COCH₂CH₂(CH₂)₁₂CH₃), 0.45-0.52 (24H, m, —COCH₂CH₂(CH₂)₁₂CH₃), 0.61-0.65 (2H, m, —COCH₂CH₂(CH₂)₁₂CH₃), 1.38 (1H, t, J=7.2 Hz, —COCH₂CH₂(CH₂)₁₂CH₃ ), 1.78 (1H, dd, J=7.0, 16.2 Hz, H-4), 1.98-2.02 (1H, m, H-4), 4.37-4.39 (1H, m, H-3), 5.07 (1H, s, H-6 or H-8), 5.13 (1H, s, H-8 or H-6), 5.83-5.87 (1H, m, H-2′), 5.91 (1H, d, J=8.0 Hz, H-3′), 5.78 (1H, s, H-6′); HR-FABMS m/z: 529.3128 ([M+H]⁺, Calcd for C₃₁H₄₅O₇: 529.3166).

### 3-O-stearoyl-(+)-catechin

(+)-catechin (1.01 g, 3.48 mmol) and stearoyl chloride (0.644 ml, 2.13 mmol) were dissolved in tetrahydrofuran (10 mL) containing trifluoroacetic acid (0.270 ml, 3.55 mmol). The solution was treated in the same way as for Example 1, yielding 3-O-stearoyl-(+)-catechin 143 mg as white powder (14.8% yield). [α]²⁰_{D}+10.4° (EtOH, c=0.5); IR (KBr) 3927, 3562, 2851, 2355, 1730, 1614, 1518, 1470, 1142, 1061, 887, 719, 598, 419 cm⁻¹; ¹H NMRδ: 0.40 (3H, t, J=6.6 Hz, —COCH₂CH₂(CH₂)₁₄CH₃), 0.75-0.88 (28H, m, —COCH₂CH₂(CH₂)₁₄CH₃), 0.94-0.97 (2H, m, —COCH₂CH₂(CH₂)₁₄CH₃), 1.71 (2H, t, J=7.4 Hz, —COCH₂CH₂(CH₂)₁₄CH₃), 2.11 (1H, dd, J=7.0, 16.6 Hz, H-4), 2.32 (1H, dd, J=5.0, 16.6 Hz, H-4), 4.70-4.73 (1H, m, H-3), 5.40 (1H, s, H-6 or H-8), 5.44 (1H, s, H-8 or H-6), 6.16-6.20 (1H, m, H-2′), 6.24 (1H, d, J=8.0 Hz, H-3′), 6.30 (1H, s, H-6′); FABMS m/z: 557.3 [M+H]⁺; HR-FABMS m/z: 557.3457 ([M+H]⁺, Calcd for C₃₃H₄₉O₇: 557.3479).

### 3-O-[(RS)-2-methyloctanoyl]-(+)-catechin

(+)-catechin (1.00 g, 3.44 mmol), (RS)-2-methyloctanoyl chloride (0.700 ml, 3.86 mmol) and trifluoroacetic acid (0.530 ml, 6.86 mmol) were dissolved in tetrahydrofuran (10 mL). The solution was treated in the same way as for Example 1, yielding 3-O-[(RS)-2-methyloctanoyl-(+)-catechin 212 mg as white powder (14.9% yield). [α]²⁰_{D}+24.6° (EtOH, c=0.8); IR (KBr) 3310, 2928, 2856, 2349, 1742, 1713, 1620, 1605, 1518, 1470, 1454, 1360, 1254, 1144, 1059, 1028, 966, 829, 731, 505 cm⁻¹; ¹H NMRδ: 0.89 (3H, t, J=6.9 Hz, —COCH(CH₃)CH₂(CH₂)₄CH₃), 0.96 (1.5H, d, J=7.0 Hz, —COCH(CH₃)CH₂(CH₂)₄CH₃), 1.00 (1.5H, d, J=6.8 Hz, —COCH(CH₃)CH₂(CH₂)₄CH₃), 1.18-1.39 (10H, m, —COCH(CH₃)CH₂(CH₂)₄CH₃), 2.27-2.35 (1H, m, —COCH(CH₃)CH₂(CH₂)₄CH₃), 2.58 (1H, dd, J=7.6, 18.4 Hz, H-4), 2.79-2.90 (1H, m, H-4), 5.17 (1H, AB, J=5.4, 7.6 Hz, H-3), 5.87 (1H, s-like, H-6 or H-8), 5.94 (1H, d, J=2.4 Hz, H-8 or H-6), 6.68 (1H, dd, J=1.9, 8.1 Hz, H-2′), 6.73 (1H, d, J=8.1 Hz, H-3′), 6.79 (1H, d, J=1.6 Hz, H-6′); FABMS m/z: 431.2 [M+H]⁺; HR-FABMS m/z: 431.2096 ([M+H]⁺, Calcd for C₂₄H₃₁O₇: 431.2070).

Therefore, it should be recognized that by modification of catechins, and especially by modifications that lead to an increased hydrophobicity (increased lipophilicity) catechins may be formed with enhanced antibacterial effect. In one exemplary modification addition of linear fatty acids to catechin (and particularly C8 and C10) enhanced the anti-staphylococcal activity of catechin against the three isolates tested. Interestingly, while certain free fatty acids (e.g., dedecanoic acid (lauric acid) (C12:0), a pahmitoleic acid isomer (C16:1delta6), and linoleic acid (C18:8)) have been reported to have anti-staphylococcal activity, free octanoic acid (C8:0) was not active against the isolates in this study. Consequently, the activity of octanoyl-(+)-catechin can not be explained by the presence of the hydrocarbon chain alone.

Remarkably, addition of a hydrophobic substituent significantly increased the bactericidal activity, both in terms of the amount of compound required to kill the bacterial cells, as well as the period of time required to achieve this. Differences in the length of time required to achieve a bactericidal affect suggests that the mechanism of killing differs between epicatechin gallate and octanoyl-(+)-catechin. While not wishing to be bound by any theory or hypothesis, the inventors contemplate that octanoyl-(+)-catechin may compromise the integrity of the cytoplasmic membrane, which may be the main antibacterial effect.

Furthermore, while previous studies on the bactericidal activity of epigallocatechin gallate by assessing the leakage of 5,6-carboxyfluorescein from liposomes have suggested that bacterial membrane damage is the mechanism of killing, possibly through interaction of ECG with phosphatidylethanolamine. Using the previous experimental conditions, ECG did appear to alter membrane permeability at concentrations 4×MIC and 8×MIC. However the degree of permeability was substantially less than for 3-octanoyl-(+)-catechin and there was little effect on cell viability for the exposure time used (10 min). Consequently, although ECG appears to initially alter the permeability of the membrane, there is still uncertainty over whether binding to the membrane per se is the lethal event.

Moreover, ECG has the capacity to modulate oxacillin resistance in S. aureus, a property not shared by catechin. Addition of hydrocarbon chains of any length did not confer the capacity to modulate oxacillin resistance on catechin. Since both acyl-(+)-catechins and ECG appear to interact with the cytoplasmic membrane, there is likely a difference in the nature of this interaction. The appearance of cells with thickened walls when grown in the presence of sub-inhibitory concentrations of ECG suggest that ECG may interfere with peptidoglycan synthesis. In contrast, Octanoyl-(−)-epicatechin did not give rise to cells with thickened cell walls but pseudomulticellular forms were noted. The gallate moiety appears to be essential for the capacity of catechins to modulate oxacillin resistance (Gallic acid itself has no anti-staphylococcal activity) or capacity to increase oxacillin susceptibility. Therefore, it should be recognized that replacement of a group in a catechin molecule (or molecule with catechin scaffold) with a lipophilic substituent will result in an enhanced antibacterial effect of such modified catechins, and especially against Staphylococcus aureus.

Thus, specific embodiments and applications of improved compositions and methods of use, for antimicrobial compositions have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A pharmaceutical composition comprising a modified catechin according to Formula 1 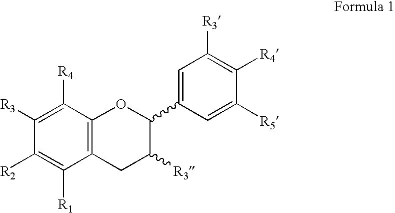
wherein R₁, R₂, R₃, R₄, R₃′, R₄′, and R₅ are independently H, OH, or M, wherein R₃″ is H, OH, an optionally substituted phenyl, or M, with the proviso that at least one of R₁, R₂, R₃, R₄, R₃′, R₄′, R₅′, and R₃″ is M;
wherein M is OC(O)R, OC(S)R, OC(NH)R, OR, or R, wherein R is optionally substituted alkyl, alkenyl, alkynyl, alkaryl, or aryl; and
wherein the modified catechin is present at a concentration effective to reduce bacterial growth in a body compartment when administered to the body compartment.

2. The pharmaceutical composition of claim 1 wherein the modified catechin has a structure according to Formula 2 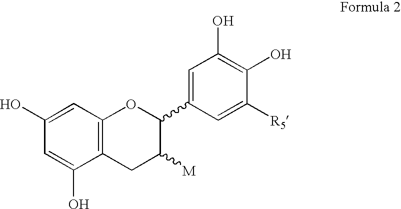
wherein R₅′ is H or OH, and wherein M is OC(O)R.

3. The pharmaceutical composition of claim 1 wherein the modified catechin has a structure according to Formula 3 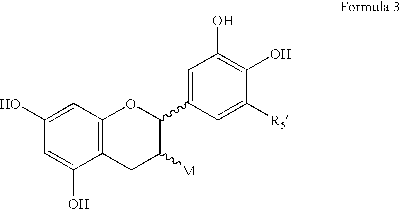
wherein M is OC(O)CH₂(CH₂)₅CH₃ or OC(O)CH₂(CH₂)₇CH₃, and R₅¹ is H or OH.

4. The pharmaceutical composition of claim 1 wherein the modified catechin has a structure according to Formula 4 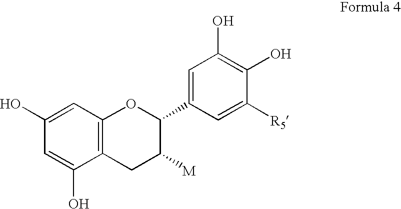
wherein M is OC(O)CH₂(CH₂)₅CH₃ or OC(O)CH₂(CH₂)₇CH₃, and R₅′ is H or OH.

5. The pharmaceutical composition of claim 1 wherein the bacterial growth is growth of a gram-positive bacterium.

6. The pharmaceutical composition of claim 5 wherein the gram-positive bacterium is Staphylococcus aureus, Streptococcus pyogenes, Streptococcus agalactiae, Propionobacterium acne, or Listeria monocytogenes.

7. The pharmaceutical composition of claim 6 wherein Staphylococcus aureus is resistant to methicillin.

8. The pharmaceutical composition of claim 1 wherein the body compartment comprises a skin of a patient and wherein the administration is topical administration.

9. The pharmaceutical composition of claim 4 wherein the body compartment comprises a skin of a patient and wherein the administration is topical administration.

10. The pharmaceutical composition of claim 1 wherein the modified catechin damages a bacterial membrane.

11. The pharmaceutical composition of claim 1 wherein the modified catechin increases sensitivity of a methicillin resistant Staphylococcus aureus towards a beta-lactam antibiotic no more than 2-fold.

12. A method of reducing growth of a bacterium comprising:
contacting the bacterium with a modified catechin having a structure according to Formula 1 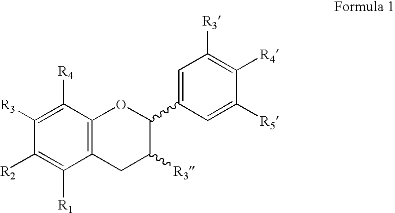
wherein R₁, R₂, R₃, R₄, R₃′, R₄′, and R₅′ are independently H, OH, or M, wherein R₃″ is H, OH, an optionally substituted phenyl, or M, with the proviso that at least one of R₁, R₂, R₃, R₄, R₃′, R₄′, R₅′, and R₃″ is M; and
wherein M is OC(O)R, OC(S)R, OC(NH)R, OR, or R, wherein R is optionally substituted alkyl, alkenyl, alkynyl, alkaryl, or aryl.

13. The method of claim 12 wherein the modified catechin has a structure according to Formula 2 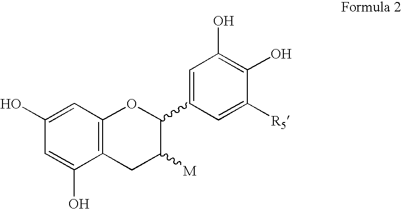
wherein R₅′ is H or OH, and wherein M is OC(O)R.

14. The method of claim 12 wherein the bacterium is a gram-positive bacterium.

15. The method of claim 14 wherein the gram-positive bacterium is Staphylococcus aureus, optionally resistant to methicillin.

16. The method of claim 14 wherein the step of contacting comprises topical application of the modified catechin to a skin of a patient infected with Staphylococcus aureus.

17. The method of claim 12 wherein the step of contacting the bacterium with the modified catechin comprises damaging a bacterial membrane.

18. A method of marketing comprising:
providing a product that includes a modified catechin according to Formula 1 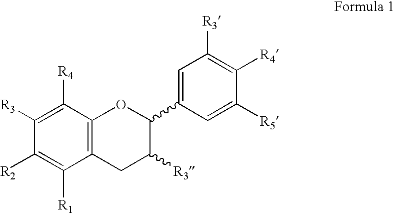
wherein R₁, R₂, R₃, R₄, R₃′, R₄′, and R₅′ are independently H, OH, or M, wherein R₃″ is H, OH, an optionally substituted phenyl, or M, with the proviso that at least one of R₁, R₂, R₃, R₄, R₃′, R₄′, R₅′, and R₃″ is M;
wherein M is OC(O)R, OC(S)R, OC(NH)R, OR, or R, wherein R is optionally substituted alkyl, alkenyl, alkynyl, alkaryl, or aryl; and
advertising that the product reduces bacterial growth.

19. The method of claim 18 wherein the product is selected from the group consisting of a cosmetic formulation, a cleaning formulation, and a pharmaceutical formulation.

20. The method of claim 18 wherein the step of advertising includes providing printed information suggesting or describing reduction of bacterial growth, or providing televised information suggesting or describing reduction of bacterial growth.
